(19) United States
(12) Patent Application Publication
(10) Pub. No.: US 2023/0227768 A1
(43) Pub. Date: Jul. 20, 2023

(54) METHOD AND SYSTEMS FOR INCREASING THE CAPACITY OF FLOW CYTOMETTER BACTERIA DETECTION AND ANTIBIOTIC SUSCEPTIBILITY TESTING SYSTEMS

(71) Applicant: Renascent Diagnostics, LLC, Burlington, VT (US)

(72) Inventors: Shawn A. Ramsaroop, St. Louis, MO (US); Matthew D. Gombrich, Underhill, VT (US)

(73) Assignee: Renascent Diagnostics, LLC, Burlington, VT (US)

(21) Appl. No.: 18/099,099

(22) Filed: Jan. 19, 2023

Related U.S. Application Data
(63) Continuation-in-part of application No. 16/779,405, filed on Jan. 31, 2020, now Pat. No. 11,579,069.
Application No. 16/779,405 is a continuation-in-part of application No. 16/096,549, filed on Oct. 25, 2018, now abandoned.
(60) Provisional application No. 62/799,488, filed on Jan. 31, 2019.
(60) Provisional application No. 62/327,007, filed on Apr. 25, 2016.
(60) Provisional application No. 62/470,595, filed on Mar. 13, 2017.

(51) Int. Cl. C12M 1/36; C12Q 1/18; C12Q 3/00; G01N 15/14

(52) U.S. Cl. CPC C12M 41/48; C12M 1/36; C12Q 1/18; C12Q 3/00; G01N 15/1425; G01N 15/1459; G01N 2015/1006

(57) ABSTRACT

A system and method for automated testing a sample of a body fluid for the presence of bacteria is described. The system includes a fluid handling device, Incubator, flow cytometer, at least a processor, and a memory configuring the at least a processor to distribute a portion of the plurality of fluid samples within a well plate to at least a first well, divide the portion of the plurality of fluid samples from the at least a first well into at least two wells including a T₀ well and a T₁ well, obtain a T₀ enumerative baseline bacterial value at time T₀, culture the fluid samples in the T₁ well using the incubator, obtain a T₁ enumerative control bacterial value at time T₁, and determine a presence of bacteria as a function of the T₀ enumerative baseline bacterial value and the T₁ enumerative control bacterial value.

### CROSS-REFERENCE TO RELATED APPLICATIONS

This application is a continuation-in-part of Non-provisional application Ser. No. 16/779,405 filed on Jan. 31, 2020, and entitled “METHODS AND SYSTEMS FOR INCREASING THE CAPACITY OF FLOW CYTOMETER BACTERIA DETECTION AND ANTIBIOTIC SUSCEPTIBILITY TESTING SYSTEMS,” which claims priority to U.S. Provisional Patent Application Ser. No. 62/799,488, filed on Jan. 31, 2019, and titled “METHODS AND SYSTEMS FOR INCREASING THE CAPACITY OF FLOW CYTOMETER BACTERIA DETECTION AND ANTIBIOTIC SUSCEPTIBILITY TESTING SYSTEMS,” and Non-provisional application Ser. No. 16/096,549 filed on Oct. 25, 2018, and entitled “SYSTEMS, DEVICES AND METHODS FOR SEQUENTIAL ANALYSIS OF COMPLEX MATRIX SAMPLES FOR HIGH CONFIDENCE BACTERIAL DETECTION AND DRUG SUSCEPTIBILITY PREDICTION USING A FLOW CYTOMETER,” which claims priority to both U.S. Provisional Patent Application Ser. No. 62/327,007, filed on Apr. 25, 2016, and titled “ANALYTICAL METHOD FOR ENUMERATIVE COMPENSATION USING A FLOW CYTOMETER,” and U.S. Provisional Patent Application Ser. No. 62/470,595, filed on Mar. 13, 2017, and titled “FLOW CYTOMETER SYSTEMS INCLUDING AUTOMATED FLUID HANDLING SYSTEMS AND METHODS OF USING THE SAME FOR QUANTIFYING THE EFFECTIVENESS OF ANTIMICROBIAL AGENTS,” the entireties of which are all incorporated herein by reference.

### FIELD OF THE INVENTION

The present invention generally relates to the field of flow cytometer bacteria detection and antibiotic susceptibility testing systems. In particular, the present invention is directed to methods and systems for increasing the capacity of flow cytometer bacteria detection and antibiotic susceptibility testing systems.

### BACKGROUND

Flow cytometer and fluid handling systems may be used for performing quantitative analyses of fluids, such as urine, blood, or cerebral spinal fluid. Any of a variety of quantitative analyses may be performed, such as detection and enumeration of one or more events of interest in a fluid sample, such as detection and enumeration of bacteria populations in a fluid sample, as well as determination of the detected bacteria population's susceptibility to one or more antibiotics. Examples of such systems and methods are disclosed in international patent application number PCT/US2017/029492, filed on Apr. 25, 2017, and titled, “Systems, Devices and Methods For Sequential Analysis Of Complex Matrix Samples For High Confidence Bacterial Detection And Drug Susceptibility Prediction Using A Flow Cytometer,” which is incorporated by reference herein in its entirety. Such automated systems can analyze a plurality of clinical samples, where the process for each sample includes a series of steps. While it is desirable to increase system throughput to maximize the number or clinical samples analyzed over a given time period, certain time periods during the analysis or a given sample, such as incubation time periods, are critical such that the system must be available to analyze a given sample at the end or such critical time periods.

### SUMMARY OF THE DISCLOSURE

In an aspect, a system for automated testing a sample of a body fluid for the presence of bacteria is described. The system includes a fluid handling device comprising a fluid handling system including an automated pipetting system configured to distribute a plurality of fluid samples within a well plate containing a plurality of wells, an incubator configured to culture the plurality of fluid samples in the well plate, a flow cytometer configured to enumerate cell counts in the plurality of fluid samples, at least a processor, and a memory communicatively connected to the at least a processor containing instructions configuring the at least a processor to distribute a portion of the plurality of fluid samples to at least a first well, divide the portion of the plurality of fluid samples from the at least a first well into at least two wells, wherein the at least two wells include a time zero (T₀) well and a time one (T₁) well, obtain a T₀ enumerative baseline bacterial value relating to fluid samples in the T₀ well at time T₀, culture the fluid samples in the T₁ well using the incubator, obtain a T₁ enumerative control bacterial value relating to fluid samples in the T₁ well at time T₁, and determining a presence of bacteria as a function of the T₀ enumerative baseline bacterial value and the T₁ enumerative control bacterial value.

In another aspect, a method for automated testing a sample of a body fluid for the presence of bacteria is described. the method includes distributing, by at least a processor, a portion of a plurality of fluid samples within a well plate to at least a first well using a fluid handling device, dividing, by the at least a processor, the portion of the plurality of fluid samples from the at least a first well into at least two wells, wherein the at least two wells include a time zero (T₀) well and a time one (T₁) well, obtaining, by the at least a processor, a T₀ enumerative baseline bacterial value relating to fluid samples in the T₀ well at time T₀ using a flow cytometer, culturing, by an incubator, the fluid samples in the T₁ well, obtaining, by the at least a processor, a T₁ enumerative control bacterial value relating to fluid samples in the T₁ well at time T₁ using the flow cytometer, and determining, by the at least a processor, a presence of bacteria as a function of the T₀ enumerative baseline bacterial value and the T₁ enumerative control bacterial value.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a functional block diagram of an automated flow cytometry and fluid handling system made in accordance with the present disclosure;

FIG. 2 is an example multi-well cassette that may be used for performing methods of the present disclosure;

FIG. 3 is an example process for analyzing a multi-well cassette for rapid determination of bacterial infection and antibiotic susceptibility;

FIG. 4 is an example timeline for sequentially processing a plurality of multi-well cassettes, each cassette containing a plurality of clinical fluid samples;

FIG. 5 is a flow chart illustrating a method of performing a sequential automated flow cytometry process on a plurality of multi-well cassettes;

FIG. 6 is a flow diagram illustrating an exemplary embodiment of a method for automated testing a sample of a body fluid for the presence of bacteria; and

FIG. 7 is a functional block diagram of an example computing system that may be used to implement aspects of the present disclosure.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Aspects of the present disclosure include methods and systems for automated analysis of clinical fluid samples, such as urine, blood, or cerebral spinal fluid, where the number of fluid samples is increased or optimized without negatively impacting the accuracy of the analysis of a given fluid sample.

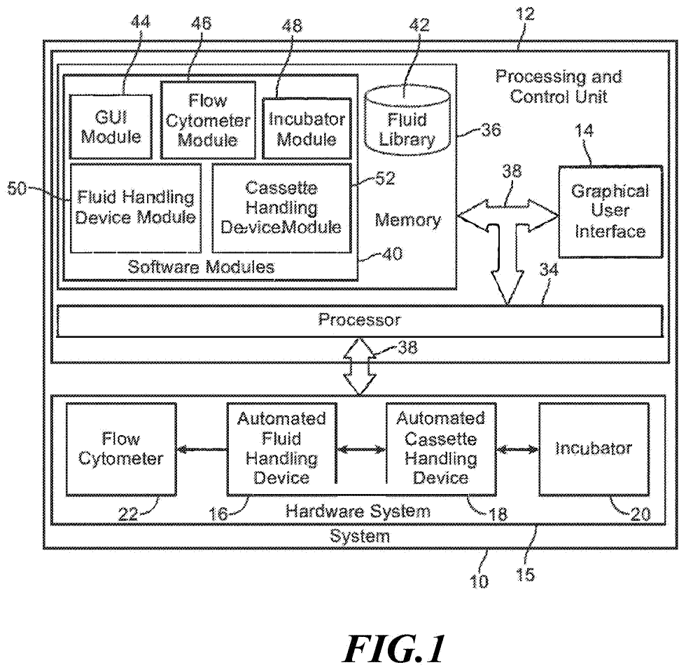

The present disclosure includes processes for loading a clinical sample in a testing well plate and using quantitative results from the initial sample testing to inform subsequent sample processing by an automated fluid handling flow cytometer system for subsequent analysis, such as antibiotic effectiveness. FIGS. 1-5 illustrate exemplary automated fluid handling flow cytometer systems, including a flow cytometer for analyzing a fluid such as fluid samples including, without limitation, urine, blood, or cerebral spinal fluid, a fluid handling system that, as described below, may be configured to provide specific concentrations of fluid mixtures at specific times to the flow cytometer for testing, a wash system for washing fluid lines in the fluid handling system between samples, a plate transport device for transporting testing well plate containing samples from an incubator to the fluid handling system, and an incubator for incubating a plurality of well plates. The system may also include a variety of software programs for operating the system.

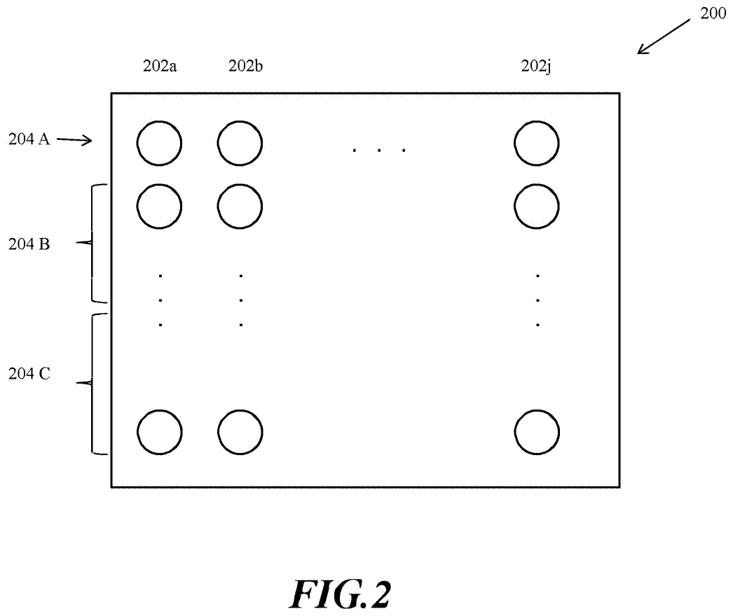

Referring now to FIG. 1, an exemplary embodiment of a flow cytometer and fluid handling system 10 is illustrated. Flow cytometer and fluid handling system 10 includes a processing and control unit 12 with a graphical user interface (GUI) 14 to allow a user to control operation of system hardware components. Hardware system 15 may include hardware components such as fluid handling system 16, automated cassette handling system 18, incubator 20 and flow cytometer 22. Memory and processor may communicate with GUI 14 and hardware system 15 through appropriate application programming interfaces (API) and communication buses 38. As described more below, flow cytometer 22 performs a variety of measurements on clinical fluid samples, however, it can only analyze one clinical sample at a time. In one example, a multi-well cassette (FIG. 2) may be used to hold multiple samples. Fluid handling system 16, automated cassette handling system 18, and incubator 20 may include one or more robotic components controlled by processors (e.g., processor 34), and may be designed and configured to automatedly transport multi-well cassettes 200 between incubator 20 and flow cytometer 22 and transport clinical samples from a given cassette to the flow cytometer for analysis. Processor 34 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 34 may include a single computing device operating independently, or may include two or more computing device operating in concert, in parallel, sequentially or the like; two or more computing devices may be included together in a single computing device or in two or more computing devices. Processor 34 may interface or communicate with one or more additional devices as described below in further detail via a network interface device. Network interface device may be utilized for connecting processor 34 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 34 may include but is not limited to, for example, a computing device or cluster of computing devices in a first location and a second computing device or cluster of computing devices in a second location. Processor 34 may include one or more computing devices dedicated to data storage, security, distribution of traffic for load balancing, and the like. Processor 34 may distribute one or more computing tasks as described below across a plurality of computing devices of computing device, which may operate in parallel, in series, redundantly, or in any other manner used for distribution of tasks or memory between computing devices. Processor 34 may be implemented using a “shared nothing” architecture in which data is cached at the worker, in an embodiment, this may enable scalability of system 100 and/or computing device.

With continued reference to FIG. 1, processor 34 may be designed and/or configured to perform any method, method step, or sequence of method steps in any embodiment described in this disclosure, in any order and with any degree of repetition. For instance, processor 34 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 34 may perform any step or sequence of steps as described in this disclosure in parallel, such as simultaneously and/or substantially simultaneously performing a step two or more times using two or more parallel threads, processor 34 cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, fluid handling system 16 may include, for example, an automated pipetting system, as well as one or more cassette handling robots and microplate washers. Automated cassette handling system 18 may be configured to transport cassettes between fluid handling system 16 and incubator 20. In some examples, automated cassette handling system 18 may be omitted and cassettes may be manually transported between fluid handling system 16 and incubator 20. As will be appreciated, the number of one or more of components in hardware system 15 may vary. For example, one or more of fluid handling system 16, auto mated cassette handling system 18, and incubator 20 may be configured to function with only one flow cytometer 22, or a plurality of flow cytometers.

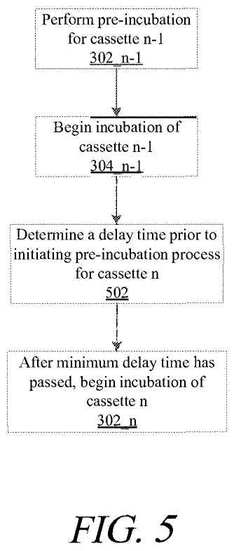

With continued reference to FIG. 1, processing and control unit 12 may include memory 36 communicatively connected to processor 34. The memory and processor communicate with GUI 14 and hardware system 15 through appropriate application programming interfaces (API) and communication buses 38. Configurations with respect to processor communication and control are described in more detail below with respect to FIG. 5. Components of memory 36 may include software modules 40 configured specifically to control and operate the connected hardware components and fluid library 42. Exemplary software modules may comprise GUI module 44, flow cytometer module 46, incubator module 48, fluid handling device module 50, and cassette handling device module 52. Fluid library 42 is populated with fluid and bacteria specific information used for analyzing the particular type of fluid under analysis, such as, but not limited to, urine, spinal fluid and blood. For example, flow cytometer software module 46 may access pre-defined regions of interest (ROIs), scatter values and fluorescence values etc. stored in the fluid library for detecting various species of bacteria in various fluids being tested. Detections in the ROI possessing characteristics of target events, such as scatter values and fluorescence values, as determined by gating strategies and/or computational analysis executed by the flow cytometer software may be used to determine concentration of particles, cells, or bacteria of interest in the sample. Also, described in more detail below, multiple ROIs for particular fluid types may be stored for use at different points of an analysis.

As will be appreciated from descriptions of various embodiments presented herein, one exemplary embodiment may comprise a kit for general bacterial staining or other analysis, including a well plate including a plurality of wells, as shown in FIG. 2. In some embodiments, well plate may include a multi-well cassette, wherein the multi-well cassette may include growth media and antibiotics in designated wells. The growth media may be provided in dried, freeze dried, or other preserved from, which may be activated, such as by hydration, in an initial processing step within or prior to placement in fluid handling device. As used herein, multi-well cassette may refer to any cassette, plate or well structure adapted for automated assaying and fluid handling. One example of such a cassette may be a 6×6 Eppendorf tube rack (and associated tubes), in which wells are formed by individual, removable tubes. Other examples of multi-well cassettes, as that term is used herein, may include conventional microwell plates, such as ninety-six well plate. Other alternative or custom multi-well cassettes may include a plurality of varying size wells for particular applications may be devised. Automated pipetting system of fluid handling device 16 may be used to distribute fluids as between wells of the well plate, such as, without limitation, multi-well cassette. fluid handling device may utilize automated pipetting system or other suitable probe to remove, e.g., aspirate, a predetermined amount of live/dead cell dye from a dye well stored in the fluid handling system and deposit the dye in the sample well containing the fluid sample. Fluid handling device may also be configured to deposit a predetermined amount of standardized fluorescent beads into the fluid sample which can be used to verify the accuracy of the flow cytometer measurements as discussed in further detail below. In other examples, dyes and beads may be manually added to the sample well.

Now referring to FIG. 2, an example multi-well cassette 200 for holding a plurality of clinical fluid samples for analysis by flow cytometer(s) 22 is illustrated. Cassette 200 includes a plurality of columns 2020-202j of wells, each column including a plurality of wells 204. In this example, “j” is a variable indicating that any number of columns may be used. In one example, separate clinical fluid samples are initially deposited in the first row (wells 204a) of each column 202 and the first row of wells are then used as a reservoir for drawing portions of the fluid sample for further processing and analysis by system 10. For example, if j=6, meaning cassette 200 includes six columns 202, six different fluid samples, e.g., urine samples from, e.g., six different patients, can be loaded into cassette 200 for automated processing.

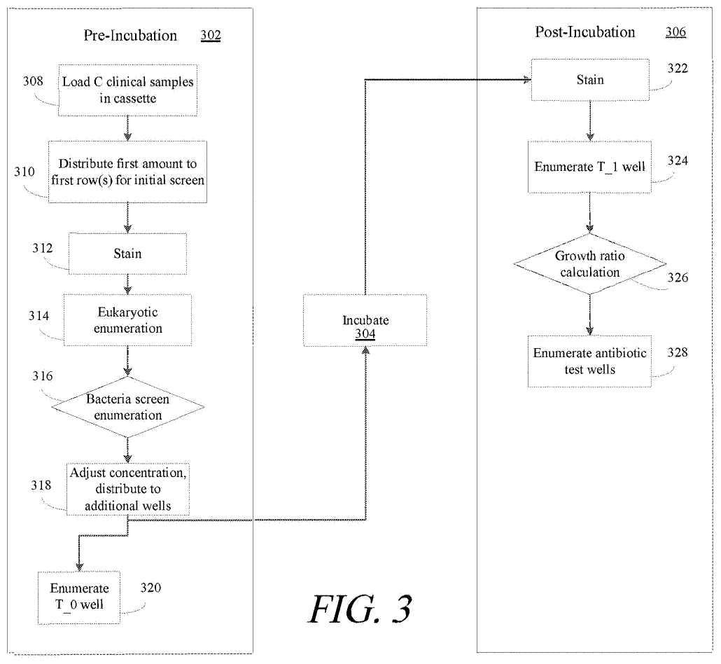

Now referring to FIG. 3, an example processes 300 for analyzing a single multi-well cassette 200 with system 10 is illustrated. In one exemplary embodiment, process 300 includes three phases, a pre-incubation phase 302, where an initial screening analysis is performed on one or more clinical samples to determine, for example, whether one or more samples contain a bacterial infection, an incubation phase 304, where one or more clinical samples are incubated for a specific period of time, and a post-incubation phase 306, where one or more samples are analyzed to verify the sample contains an infection of pathogenic bacteria and to identify one or more antibiotics that may be effective in combating the pathogenic bacteria population(s).

With continued reference to FIG. 3, pre-incubation phase 302 may begin at step 308, with a single volume of a sample being loaded in the first row 204a of cassette 200. For a cassette containing j columns of wells, j samples may be loaded in the first row 204a of corresponding respective columns 202a-j. Cassette 200 may have a predetermined volume of growth media, e.g., 1 ml, in one or more media wells. In one example, Mueller Hinton Broth may be used as the growth media. Fluid handling system 16 may contain one or more wells, volumes, or containers, with dyes, staining agents, control beads, and antibiotics for use during an automated analysis process.

With continued reference to FIG. 1, Processor 34 is configured to distribute a portion of plurality of fluid samples to at least a first well using fluid handling device described above. The distribution of samples may include an equal distribution. This includes a T₀ control well, T₁ control well, and all wells containing antibiotics. Plurality of fluid samples may be loaded onto the automated flow cytometer and the sample ID may be assigned to the well plate. During use, processor 34 is configured to divide fluid sample among plurality of wells of the well plate. Fluid sample may be treated with a staining reagent that stains at least live bacteria, and in some cases, also stains dead cells to differentiate between live and injured/dead bacteria cells. One or more antimicrobial agents can be selected and added to various wells to test the efficacy of the antimicrobial agents for treating any bacteria that is present. There may be at least two control wells (T₀, and T₁ wells that contain growth media but no antimicrobial agent) and in some cases at least one additional well for testing an antimicrobial agent.

With continued reference to FIG. 3, at step 310, after j samples are loaded in row 204a, fluid handling system 16 may utilize automated pipetting system or other suitable probe to remove, e.g., aspirate, a predetermined amount of each sample to row(s) in row group 204b for pre-incubation analysis. In one example, row group 204b includes two rows. At step 312, fluid handling system 16 may obtain appropriate cellular stains from designated positions in the fluid handling system and stain the fluid samples in rows 204b. In some embodiments, the dyes may include at least two different dyes, for example one dye that permeates only dead cells, e.g., propidium iodide, and another that permeates all cells, e.g., thyzol orange. Using distinct dye types in this manner allows for discrimination between live and dead cells based on the different fluorescence characteristics of the different dyes when interrogated by appropriate excitation light sources(s).

With continued reference to FIG. 3, at step 310, after j samples are loaded in row 204a, fluid handling system 16 may utilize automated pipetting system or other suitable probe to remove, e.g., aspirate, a predetermined amount of each sample to row(s) in row group 204b for pre-incubation analysis. In one example, row group 204b includes two rows. At step 312, fluid handling system 16 may obtain appropriate cellular stains from designated positions in the fluid handling system and stain the fluid samples in rows 204b. In some embodiments, the dyes may include at least two different dyes, for example one dye that permeates only dead cells, e.g., propidium iodide, and another that permeates all cells, e.g., thyzol orange. Using distinct dye types in this manner allows for discrimination between live and dead cells based on the different fluorescence characteristics of the different dyes when interrogated by appropriate excitation light sources(s).

With continued reference to FIG. 3, at step 314, fluid handling system 16 may then deliver the contents of a first well, e.g., a first row 204b of a first column 202a to flow cytometer 22 for a first analysis, e.g., eukaryotic enumeration. Distributing the portion of plurality of fluid samples may include determining a total bacteria count of the portion of the plurality samples by enumerating the portion of the plurality of fluid samples using the flow cytometer. In a non-limiting example, flow cytometer may be configured to perform a eukaryotic enumeration. The analysis may include scatter plots and fluorescence plots that include gates for red and white blood cell counts. This analysis enables accurate enumeration of specific cell populations that may provide clinically relevant information for the disease process being screened. As an example, the presence of white blood cells in urine samples being screened for urinary tract infections is a secondary indicator of active infection, above and beyond the presence of bacteria. In some embodiments, total bacteria count may exclude dead cells depending on techniques employed.

With continued reference to FIG. 3, in an embodiment, next, at step 316, contents of one sample column 202 in a second row in row group 2046 are delivered by fluid handling system 16 to flow cytometer 22 for bacteria screen enumeration. Scatter plot gating and fluorescence intensity analysis may again be used to deter a bacteria count corresponding to events falling within an ROI. The bacteria screen count of step 316 may utilize the live/dead cell staining applied at step 312 to exclude dead cells from the bacteria enumeration. The live bacteria cell enumeration can be compared to predetermined threshold values to assess whether continued analysis of the sample is warranted. For example, current clinical standards relative to assessment of urinary tract infections indicate thresholds of 10⁴/ml or 10⁵/ml depending on factors such as clinical status of the patient. Other threshold values may be applied as appropriate for analysis of other clinical indications or other clinical situations. Persons of ordinary skill will appreciate that in other embodiments the enumerations of samples may be performed in reverse order, or, alternatively, to the extent not excluded by hardware or system limitations, simultaneously performed.

With continued reference to FIG. 3, it should be noted that while the bacteria screen step 316 may be conducted to largely eliminate dead cells from the cell count based on use of fluorescence discriminating dyes, cell count at this stage may still include all types of live cells, both live cells of interest and live cells that are not of interest that may thus be considered as contaminant cells. For example, in assessment of urinary tract infections, a primary pathogenic bacteria of interest is E. coli. However, a typical human urine sample may also include many different species of non-pathogenic flora. These non-pathogenic flora may be considered as contaminants with respect to accurate clinical analysis of pathogens. Thus, after completion of step 316 system 10 may stop analyzing samples in one or more of columns 202. For example, the bacteria count determined at step 316 for one or more of the samples initially loaded in columns 202a-j may have a bacteria count below the applicable threshold, indicating the sample does not meet a clinical definition of a bacterial infection and does not require additional processing and analysis.

With continued reference to FIG. 3, based on bacterial count determined in the preceding steps, in step 318 sample concentration is adjusted to a target bacterial level and samples distributed from row 204a to row group 204c, such as, without limitation, T₀ well and T₁ well described in further detail below, for further analysis. Adjustment of sample concentration may be accomplished by addition of appropriate amounts of growth media when samples are further distributed by automated pipetting system 24. Sample distribution will include at least distribution of the T₀ sample to a first well and the T₁ sample to a second well. First well may include wells from a first row of well plate and second well may include wells from a second row of well plate. Optionally, further samples may be distributed to antibiotic testing (AT) wells in group 204c if antibiotic testing is to be included in the analysis. In one example, this step is omitted for any sample(s) that system 10 determined at step 316 did not contain a live bacteria count above the applicable threshold. As is known in the art, testing of bacteria for antibiotic resistance or susceptibility typically requires a bacterial concentration in the range of approximately 1×10⁵ to approximately 1×10⁶ bacteria/ml. However, depending on the sensitivity and accuracy of the instrumentation employed (for example some flow cytometer systems are more sensitive than others), lower concentrations may be employed. Thus, methods of the present disclosure may be employed with concentrations as low as in the range of 1×10³ bacteria/ml. For example, instrument sensitivity may indicate a concentration in the range of approximately 1×10⁴ bacteria/ml to approximately 5×10⁴ bacteria/ml, or other instrumentation may employ a concentration in the range of approximately 1×10³ bacteria/ml to approximately 5×10³ bacteria/ml. Various antimicrobial efficacy testing methods may require a standard concentration of bacteria, e.g., a predetermined bacterial concentration of 1×10⁵ bacteria/ml.

With continued reference to FIG. 3, if initial testing of a clinical sample indicates a higher concentration, e.g., if the flow cytometer enumerates an initial sample at 1×10⁷ bacteria/ml, the system may automatically adjust the concentration for sub sequent testing. In one example, 1 microliter of the sample may be aspirated by the fluid handling system and deposited into 1000 microliters of media in a first one of media wells 204c to arrive at the target concentration of 1×10⁴. In another example, the initial concentration may be greater than 1×10⁷ bacteria/ml, and/or the minimum aspiration volume may be greater than 1 microliter, and/or the target concentration may be lower, etc. such that a second dilution step is required. The fluid handling system may be configured to determine a second amount of fluid to be aspirated from the first media well containing media and the first amount of the fluid sample for deposit in a second media well in group 204c to arrive at the target concentration, e.g., 1×10⁴ bacteria/ml.

With continued reference to FIG. 3, sample distribution at step 318 includes distribution of a time zero control, T0, sample to a first well in group 204c as well as a T1 sample to a second well in group 204c. In one embodiment, adjustment step 318 is accomplished by depositing a properly diluted sample in an initial well in group 204c and then distributing an amount of the properly-diluted sample from the initial well to all other wells to be employed. T₀ well sample, as well as T₁ well sample, may be analyzed with a flow cytometer that includes a bacteria library that defines a region of interest (ROI) tailored to bacteria. Samples such as fluid samples in T₀ well may be tested at T₀ to obtain T₀ enumerative baseline bacterial value for any population assigned to the bacterial region of interest (ROI), including enumeration, mean fluorescence, CV, range of the population, and if fluorescent compensation beads are used, a calculation of a distance from the compensation beads in the sample. This data is stored and used as a reference for subsequent analysis after sample incubation at time one T₁. Subsequent volumes of the sample such as, without limitation, fluid samples in T₁ well, may be incubated at a pre-determined time and temperature (e.g., 37 C) and analyzed by the system at time T₁, and perhaps beyond. Comparative analysis to the initial sample in T₀ well would ensue to determine population expansion in the pre-determined bacteria-specific ROI. Time T₀ and time T₁ may be determined by processor 34 as described below in this disclosure.

With continued reference to FIG. 3, at step 320, a first sample, referred to herein as a T0 sample (i.e., fluid samples in T₀ well), from the properly-diluted samples in group 204c, is transported to flow cytometer 22 to obtain a T₀ enumerative baseline bacterial value. As used in this disclosure, “T₀ enumerative baseline bacterial value” is a bacteria count of fluid samples in T₀ well at time zero T₀. In a non-limiting example, processor 104 may be configured to enumerating, using flow cytometer, fluid samples in T₀ well at time T₀ to obtain T₀ enumerative baseline bacterial value. After removing a portion of the T0 sample from cassette 200 for enumeration, at step 304 the cassette 200 containing a second sample for enumeration after incubation, the T1 sample (i.e., fluid samples in T₁ well), and any desired antibiotic testing samples is delivered to incubator 20 by automated cassette handling system 18 and incubated. Processor 34 is configured to culture the fluid samples in T₁ well using incubator 20. Culturing the fluid samples in T₁ well may include delivering, using a plate transport device such as automated cassette handling device 18, the fluid samples in the T₁ well to incubator 20 using and returning, using the plate transport device, the fluid samples from the incubator to the fluid handling device 16 after culturing.

AT wells in group 204c may be prefilled with specific antibiotics against which testing is to be run or may be separately filled from an appropriate source by the fluid handling system. Incubation time will depend on the nature of the cells to be studied. For example, with respect to cells of interest, such as urogenital flora, incubation time may be in the range of about 2.5 hours, or typically less than about 3 hours, but more than 2 hours. As described more below, in some examples, it can be very important that each cassette 200 containing the same type of fluid sample is incubated for the same period of time.

With continued reference to FIG. 3, after incubation, at step 322, the multi-well cassette is returned to fluid handling system 16 by automated cassette handling system 18. At step 322, all T1 samples and AT wells in group 204c are stained by fluid handling system 16. In one example, the same live/dead stains that were used in step 312 are used here. Thereafter, at step 324, processor 34 is configured to obtain a T₁ enumerative control bacterial value relating to T1 samples. As used in this disclosure, “T₁ enumerative baseline bacterial value” is a bacteria count of fluid samples in T₁ well at time one T₁. In a non-limiting example, processor 34 may be configured to enumerate the fluid samples in T₁ well at time T₁ to obtain T₁ enumerative baseline bacterial value. Processor 34 is further configured to determine a presence of bacteria as a function of T₀ enumerative baseline bacterial value and the T₁ enumerative control bacterial value. In a non-limiting example, determining the presence of bacteria may include comparing the T₁ enumerative control bacterial value to the T₀ enumerative baseline bacterial value and determining a growth ratio of the portion of the plurality of fluid samples as a function of the comparison; for instance, and without limitation, growth ratio may include a ratio of T1 to T0 cells, determined at step 326. Enumeration (316, 324) and assessment of the T1/T0 cell growth ratio (326) are important steps to allow quantitative discrimination between pathogenic cells/bacteria of interest and contaminant cells/bacteria. It has been determined by the Applicant that pathogenic bacteria exhibit different growth rates as compared to non-pathogenic, contaminant bacteria and that these differences in growth rate may be used to discriminate qualitatively between cells of clinical interest and contaminant cells, without reliance on more subjective measures such as species identification using chemical means or matrix assisted laser desorption/ionization time of flight mass spectrometry (MALDI-TOF). For example, it has been determined that pathogenic cells in human urine exhibit a growth rate that is approximately 5x±1 greater than the growth rate of contaminant cells when cultured over short culture times in the range of approximately 2.5 hours. It may be possible in certain circumstances to state the growth rate difference more specifically as 5x±0.5. Thus, in one embodiment, if the T1 to T0 cell growth ratio is determined to be between about 6.25x and 16.25x (i.e., about 125% to about 325%) the sample may be assessed as a positive for pathogenic bacteria.

With continued reference to FIG. 3, in another embodiment, the system may be programed to convert the relative growth between T0 and T1 to an integer representing bacterial population expansion. In such an embodiment, the derived growth integer from T0 baseline to T1 control growth is compared to the known growth integers of a known library of pathogens represented in the disease state being tested. Representative disease states may include, but are not limited to, pathogens associated with urinary tract infections, pathogens associated with blood stream infections (bacteremia/sepsis), pathogens associated with meningitis or other neurologic infections. Alternatively, or additionally, the derived growth integer is compared to the known growth integers of a known library of possible bacterial contaminants represented in the disease state being assessed, such as, but not limited to normal urogenital flora associated with suspected urinary tract infections or possible skin contaminant associated with blood sampling in suspected bacteremia samples. Known libraries of pathogens and contaminants may be stored in fluid library 42 in memory 36.

With continued reference to FIG. 3, depending on the clinical objective, for example if simply determining existence of a urinary tract infection is the goal, then the positive result may be the stopping point and the result reported to the appropriate health care provider or patient. However, embodiments of the present disclosure also provide for rapid assessment of antibiotic resistance/susceptibility prediction if such information is desired. If the result of the assessment in step 326 is positive, enumeration of the samples placed in the AT wells may proceed. Because the samples were distributed to the AT wells at the same time as the T0 and T1 wells, the samples in the AT wells were cultured also during incubation step 304 and thus may be immediately enumerated without additional culture time. At step 328, samples from AT wells in group 204c for each of columns 202 that tested positive at steps 316 and 326 are enumerated to determine an antibiotic prediction profile or for use as information in determining antibiotic susceptibility based on comparison with the T1 sample. For these comparisons, the T1 enumeration provides a baseline against which the AT enumeration is compared. Resistance prediction may be based on growth rate thresholds as may be established for specific clinical indications and/or drugs and antibiotics. Note that once again, by using flow cytometer enumeration and comparing the ratio of, e.g., ATn/T1, a quantitative measurement of the antibiotic/drug effectiveness may be determined.

With continued reference to FIG. 3, automated flow cytometry systems made in accordance with the present disclosure can be configured to process a plurality of multi-well cassettes, such as multi-well cassette 200, each of which may contain a plurality of different fluid samples. As described above in connection with FIG. 3, the analysis of each cassette includes three phases a pre-incubation phase 302, an incubation phase 304, and a post-incubation phase 306. After system 10 performs pre-incubation phase 302 on a first cassette and the first cassette is deposited in incubator 20, the first cassette will need to remain in the incubator for a relatively long time, e.g., three hours. System 10 can, therefore, begin the pre-incubation phase 302 for a second cassette, however, as noted above, it is important that post-incubation phase 306 begins substantially immediately after reaching the required incubation time because the growth ratio calculations per formed at steps 326 and 328 and determinations of infection and antibiotic effectiveness are based on a pre-determined incubation time and temperature.

With continued reference to FIG. 3, the enumerative accuracy of the bacteria-specific region at all testing times is improved by utilizing a test enumerative compensator (TEC) particle, such as a bead with flow cytometric scatter and fluorescence characteristics similar, yet slightly altered from the target population, within the test run with known expected values with the aim of compensating data acquisition deficiencies during the test run. A unique ROI separate from the live bacteria ROI may be created to enumerate the TEC during every analysis run. Additionally, at least two batches further include n AT samples, each one of the n AT samples being treated by a different one of n different antibiotics, wherein n is an integer greater than zero, the method further including testing each of the n AT samples at time T₁ with the flow cytometer to obtain n AT sample values; comparing the T₀ control live cell events in the ROI to each of the n T₁ sample live cell events in the ROI to determine the susceptibility or resistance of detected bacteria to the n different antibiotics. As desired, all AT samples tested for bacterial enumeration values may be compensated using TEC compensator particles according to the method described above. In a non-limiting example, processor 34 may be further configured to adjust a test enumerative bacterial value as a function of a compensator factor, wherein adjusting the test enumerative bacterial value may include including a known concentration of a test-enumerative compensator (TEC) particles in the fluid sample to be enumerated, wherein the TEC particles may include known flow cytometric scatter and fluorescence characteristics, enumerating the TEC particles with the sample enumeration by the flow cytometer 22, and determining the compensator factor as a function of a comparison of a test enumerative bacterial value of the TEC particles to the know concentration of the TEC particles.

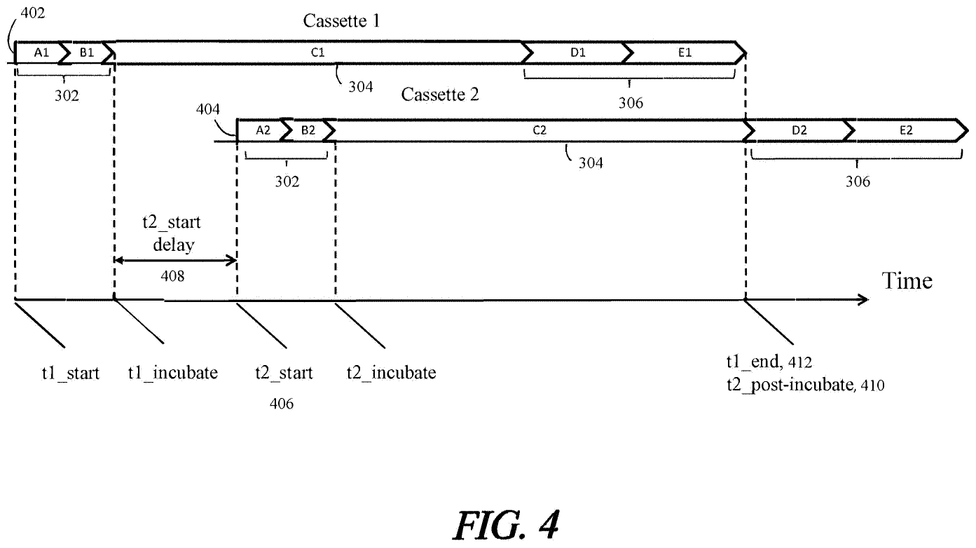

Now referring to FIG. 4, such time dependency between the analysis of sequential cassettes is illustrated, which shows a timeline 402 for analysis of a first cassette and a timeline 404 for analysis of a second cassette. Pre-incubation phase 302 includes a first period A that represents a portion of the pre-incubation phase through an initial live bacteria enumeration, e.g., steps 308-316. Second period B, which is the amount of time required to perform a pre-incubation process on x clinical samples of a cassette after an initial live bacteria enumeration, e.g., steps 318-320. As noted above, after the initial bacteria screen at step 316, system 10 may be configured to only continue to process the samples that have a live bacteria count that exceeds a pre-determined threshold, such that time period B of pre-incubation phase 302 may vary from cassette to cassette.

With continued reference to FIG. 4, timelines 402, 404 also include the incubation phase C1 304, and contain post-incubation phase 306, which include a first period, D, which represents the amount of time after incubation through performing a growth ratio determination process, e.g., steps 322-326. Post-incubation phase 306 may also include a second period, E, which is the amount of time required to perform a bacteria susceptibility determination process, e.g., step 328. As noted above, in some examples, system 10 may be configured to only perform step 328 to analyze the AT wells for samples that meet or exceed a threshold ratio determined in step 326.

With continued reference to FIG. 4, as showed conceptually, system 10 may need to delay the start of the second cassette, t2_start 406 by a delay time t2_start delay 408 to ensure the beginning of post-incubation phase 306 for cassette 2 (t2_post-incubate 410) does not occur prior to the end of post-incubation phase 306 for cassette 1 (t1_end 412). Incorporating any required delay prior to analysis of cassette 2 ensures flow cytometer 22 has completed the post-incubation phase 306 for a first cassette and is available to begin the post-incubation phase 306 of a second cassette. As noted above, this can be important for ensuring the accuracy and reliability of the measurements and analytical results for the second cassette. As will be appreciated, FIG. 4 is a simplified conceptual illustration of only two cassettes, however, system 10 can be configured to concurrently process a significantly greater number of multi-well cassettes, with a plurality of the cassettes undergoing incubation phase 304 in incubator 20 at the same time. The relationship illustrated in FIG. 4 applies to any two sequential cassettes. Also, the relative durations of the phases illustrated in FIG. 4 are not drawn to scale. For example, incubation phase 304 may be a longer duration relative to pre and post incubation 302, 306. Also, as noted above, at least time periods B, D, and E may vary from cassette to cassette, depending on the number of clinical samples that test positive for a bacterial infection.

Now referring to FIG. 5, a method of sequentially performing the automated flow cytometry process of FIG. 3 on two multi-well cassettes is illustrated. At step 302_n−1 the pre-incubation process steps 302 (FIG. 3) are performed on a first cassette n−1. At step 304_n−1, the incubation of first cassette n−1 begins, and at step 502, a delay time prior to initiating the pre-incubation process 302 for a subsequent cassette n is determined. At step 302_n, after the required delay time after incubation of cassette n−1 has passed, the pre-incubation process 302_n for cassette n begins.

With continued reference to FIG. 5, processor 34 may be configured to execute one or more calculations in connection with performing step 502 of FIG. 5—determination of a delay time, as well as other delay times as described below. In one example, calculations for determining a delay in a start time for analysis of a given multi-well cassette, n, may involve one or more of Equations (1)-(6) as follows:

t_{delay}_{ₙ}>t_{post-incubation,n-1}−t_{pre-incubation,n} Eq. (1)

Wherein t_{delay}_{ₙ} is the minimum required time delay prior to beginning a first step, e.g., step 308 of an automated flow cytometry process of a cassette, n, after an incubation period of a previously-analyzed cassette, n−1, begins; t_{post-incubation,n-1} is the amount of time required to complete post incubation processes, e.g., steps 322-328, after an incubation, e.g., step 304 of previously analyzed cassette, n−1; and t_{pre-incubation,n} is the amount of time required to complete pre-incubation processes, e.g., steps 308-320.

t_{post-incubation,n-1}(x, y)=t_{D,n-1}(x)+t_{E,n-1}(y) Eq. (2)

Wherein t_{D,n-1}(x) is the amount of time required to perform a growth ratio determination process, e.g., steps 322-326; and t_{E,n-1}(y) is the amount of time required to perform a bacteria susceptibility determination process, e.g., step 328.

t_{D,n-1}(x)=j+k×x Eq. (3)

Wherein j is a constant, in some examples, about 5 to 15 minutes, and in some examples, about 12 minutes; k is a constant, in some examples, about 1 to 3 minutes, and in some examples about 1.25 minutes; and x is the number of clinical samples containing a concentration of live bacteria above a threshold value, determined during a pre-incubation live bacteria enumeration process, e.g., step 316.

t_{E,n-1}(y)=l+m×y Eq. (4)

Wherein l is a constant, in some examples, about 5 to 10 minutes, and in some examples, about 8 minutes; m is a constant, in some examples, about 5 to 10 minutes, and in some examples about 7 minutes; and [0048] y is the number of clinical samples containing bacteria population(s) having a rate of bacteria population expansion during an incubation period that exceeds a threshold value, determined during a post incubation live bacteria enumeration process and comparison to a pre-incubation bacteria enumeration, e.g., step 326.

t_{pre-incubation,n}(c, x)=t_{A,n}(C)+t_{B,n}(x) Eq. (1)

wherein t_{A,n}(C) is the amount of time required to perform a pre-incubation process through an initial live bacteria enumeration, e.g., steps 308-316; t_{B,n}(x) is the amount of time required to perform a pre-incubation process on x clinical samples of a cassette after an initial live bacteria enumeration, e.g., steps 318-320; c is the number of clinical samples that can be loaded on a cassette; and x is the number of clinical samples containing a concentration of live bacteria above a threshold value, determined during a pre-incubation live bacteria enumeration process, e.g., step 316.

t_{B,n}(x)=n+o×x Eq. (1)

wherein n is a constant, in some examples, about 11 to 20 minutes, and in some examples, about 35 minutes; o is a constant, in some examples, about 13 to 30 minutes, and in some examples, about 50 minutes; and x is the number of clinical samples containing a concentration of live bacteria above a threshold value, determined during a pre-incubation live bacteria enumeration process, e.g., step 316.

With continued reference to FIG. 5, thus, as described above, the minimum required time delay before commencing pre-incubation phase 302 is a function of the duration of the pre-incubation phase for that cassette and the post-incubation phase 306 for the previously-analyzed cassette. As noted above, the time duration of the post-incubation phase is a function of the number of clinical samples contained on the cassette that tested positive in the initial screening step 316, and the number of samples that tested positive in the growth ratio calculation step 326 (FIG. 3). Thus, the minimum required time delay for cassette n increases as the number of clinical samples on cassette n−1 containing a bacterial infection increase. As will be appreciated, Equation (1) represents a minimum time delay and a longer time delay prior to commencement of analysis of a subsequent cassette may be used. Further, the example described above assumes a constant incubation time for all cassettes, however, Equations (1)-(6) can be readily modified to incorporate a variable incubation time, which may be applicable when cassettes with differing types of fluids, e.g., urine, blood, and/or cerebral spinal fluid, are being analyzed by system 10 at the same time. In another example, system 10 may incorporate two time delays. For example, the initial time delay t_{delay,n}, may assume a nominal number of samples on cassette 200 will test positive in screening step 316. As illustrated in Equations (1), (5), and (6), if the assumption over-predicts the number of infected samples, the time duration of the pre-incubation phase will be shorter, requiring a longer minimum time delay t_{delay,n}. A second time delay may be incorporated prior to commencing with step 318 to account for the over-prediction to ensure cassette n does not begin incubation too soon.

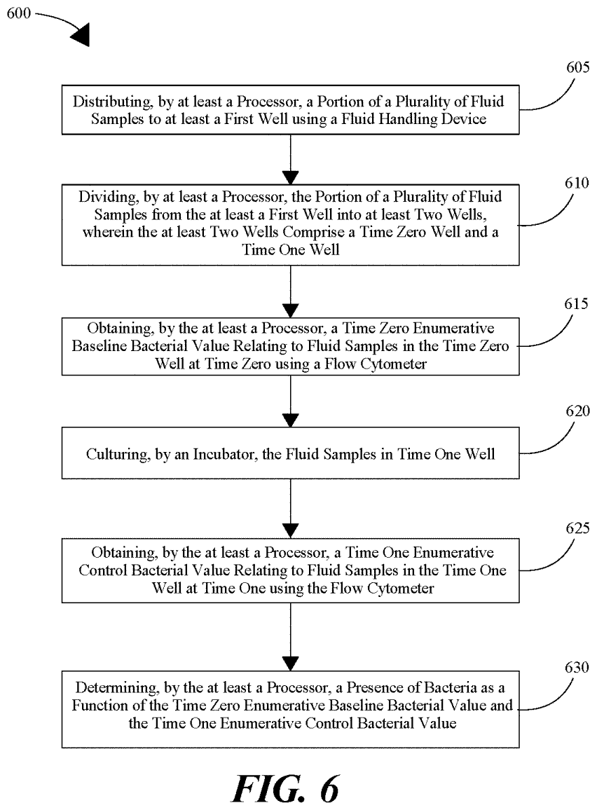

Now referring to FIG. 6, an exemplary method 600 for automated testing a sample of a body fluid for the presence of bacteria is illustrated. Method 600 includes a step 605 of distributing, by at least a processor, a portion of the plurality of fluid samples to at least a first well within a well plate containing a plurality of wells using a fluid handling device. This may be implemented, without limitation, as described above in reference to FIGS. 1-5. In some embodiments, the well plate may include a multi-well cassette. In some embodiments, distributing the portion of the plurality of fluid samples to the at least a first well may include determining a total bacteria count of the portion of the plurality of fluid samples by enumerating the portion of the plurality of fluid samples using the flow cytometer. In some embodiments, distributing the portion of the plurality of fluid samples to the at least a first well may include adjust a dilution of the portion of the plurality of fluid samples with a growth media to a predetermined connection as a function of the total bacteria count. This may be implemented, without limitation, as described above in reference to FIGS. 1-5.

With continued reference to FIG. 6, method 600 includes a step 610 of dividing, by the at least a processor, the portion of the plurality of fluid samples from the at least a first well into at least two wells, wherein the at least two wells comprise a time zero (T₀) well and a time one control (T₁) well. This may be implemented, without limitation, as described above in reference to FIGS. 1-5.

With continued reference to FIG. 6, method 600 includes a step 615 of obtaining, by the at least a processor, a T₀ enumerative baseline bacterial value relating to fluid samples in the T₀ well at time T₀ using a flow cytometer. This may be implemented, without limitation, as described above in reference to FIGS. 1-5. In some embodiments, obtaining the T₀ enumerative baseline bacterial value may include enumerating the fluid samples in the T₀ well at time T₀. This may be implemented, without limitation, as described above in reference to FIGS. 1-5.

With continued reference to FIG. 6, method 600 includes a step 620 of culturing, by an incubator, the fluid samples in the T₁ well, without limitation, as described above in reference to FIGS. 1-5. In some embodiments, culturing the fluid samples in T₁ well may include delivering, using the plate transport device, the fluid samples in the T₁ well to the incubator and returning, using the plate transport device, the fluid samples from the incubator to the fluid handling device after culturing. This may be implemented, without limitation, as described above in reference to FIGS. 1-5.

With continued reference to FIG. 6, method 600 includes a step 625 of obtaining, by the at least a processor, a T₁ enumerative control bacterial value relating to fluid samples in the T₁ well at time T₁ using the flow cytometer. This may be implemented, without limitation, as described above in reference to FIGS. 1-5. In some embodiments, obtaining the T₁ enumerative control bacterial value may include enumerating the fluid samples in the T₁ well at time T₁. This may be implemented, without limitation, as described above in reference to FIGS. 1-5.

With continued reference to FIG. 6, method 600 includes a step 630 of determining, by the at least a processor, a presence of bacteria as a function of the T₀ enumerative baseline bacterial value and the T₁ enumerative control bacterial value. This may be implemented, without limitation, as described above in reference to FIGS. 1-5. In some embodiments, determining the presence of bacteria may include comparing the T₁ enumerative control bacterial value to the T₀ enumerative baseline bacterial value and determining a growth ratio of the portion of the plurality of fluid samples as a function of the comparison. This may be implemented, without limitation, as described above in reference to FIGS. 1-5.

With continued reference to FIG. 6, method 600 may further include a step of adjusting a test enumerative bacterial value as a function of a compensator factor. This may be implemented, without limitation, as described above in reference to FIGS. 1-5. In some embodiments, adjusting the test enumerative bacterial value may include including a known concentration of a test-enumerative compensator (TEC) particles in the fluid sample to be enumerated, wherein the TEC particles may include known flow cytometric scatter and fluorescence characteristics, enumerating the TEC particles with the sample enumeration by the flow cytometer, and determining the compensator factor as a function of a comparison of a test enumerative bacterial value of the TEC particles to the know concentration of the TEC particles. This may be implemented, without limitation, as described above in reference to FIGS. 1-5.

As will be appreciated, one or more of software modules 40 may include machine executable instructions, executable by processor 34, for automatically determining any required time delays prior to processing a multi-well cassette, which may involve accessing the results from one or more of steps 316, 324 and 326, which may be stored in memory 36 and for otherwise coordinating the parallel processing of a plurality of multi-well cassettes 200 with one or more flow cytometers 22. It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

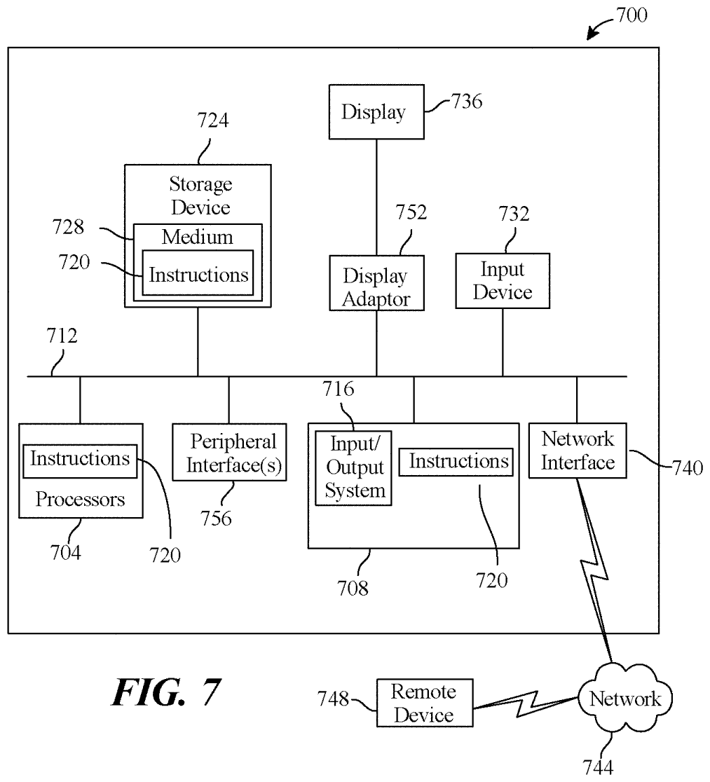

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for automated testing of a sample of a body fluid for the presence of bacteria, the system comprising:
a fluid handling device comprising a fluid handling system, wherein the fluid handling system comprises an automated pipetting system configured to distribute a plurality of fluid samples within a well plate comprising a plurality of wells;
an incubator configured to culture the plurality of fluid samples in the well plate;
a flow cytometer configured to enumerate cell counts in the plurality of fluid samples;
at least a processor; and
a memory communicatively connected to the at least a processor, the memory containing
instructions configuring the at least a processor to:
distribute a portion of the plurality of fluid samples to at least a first well;
divide the portion of the plurality of fluid samples from the at least a first well into at least two wells, wherein the at least two wells comprise:
a time zero (T₀) well; and
a time one (T₁) well;
obtain a T₀ enumerative baseline bacterial value relating to fluid samples in the T₀ well at time T₀;
culture the fluid samples in the T₁ well using the incubator;
obtain a T₁ enumerative control bacterial value relating to fluid samples in the T₁ well at time T₁; and
determine a presence of bacteria as a function of the T₀ enumerative baseline bacterial value and the T₁ enumerative control bacterial value.

2. The system of claim 1, wherein the well plate comprises a multi-well cassette.

3. The system of claim 1, wherein distributing the portion of the plurality of fluid samples to the at least a first well comprises determining a total bacteria count of the portion of the plurality of fluid samples by enumerating the portion of the plurality of fluid samples using the flow cytometer.

4. The system of claim 3, wherein distributing the portion of the plurality of fluid samples to the at least a first well comprises adjusting a dilution of the portion of the plurality of fluid samples using a growth media to a predetermined concentration as a function of the total bacteria count.

5. The system of claim 1, wherein obtaining the T₀ enumerative baseline bacterial value comprises enumerating the fluid samples in the T₀ well at time T₀.

6. The system of claim 1, wherein culturing the fluid samples in the T₁ well comprises:
delivering, using a plate transport device, the fluid samples in the T₁ well to the incubator; and
returning, using the plate transport device, the fluid samples from the incubator to the fluid handling device after culturing.

7. They system of claim 1, wherein obtaining the T₁ enumerative control bacterial value comprises enumerating the fluid samples in the T₁ well at time T₁.

8. The system of claim 1, wherein determining the presence of bacteria comprises:
comparing the T₁ enumerative control bacterial value to the T₀ enumerative baseline bacterial value; and
determining a growth ratio of the portion of the plurality of fluid samples as a function of the comparison.

9. The system of claim 1, wherein the memory further contains instructions configuring the at least a processor to adjust a test enumerative bacterial value as a function of a compensator factor.

10. The system of claim 9, wherein adjusting the test enumerative bacterial value comprises:
including a known concentration of a test-enumerative compensator (TEC) particles in the fluid sample to be enumerated, wherein the TEC particles comprise known flow cytometric scatter and fluorescence characteristics;
enumerating the TEC particles with the sample enumeration by the flow cytometer; and
determining the compensator factor as a function of a comparison of a test enumerative bacterial value of the TEC particles to the know concentration of the TEC particles.

11. A method for automated testing a sample of a body fluid for the presence of bacteria, the method comprises:
distributing, by at least a processor, a portion of a plurality of fluid samples within a well plate to at least a first well using a fluid handling device;
dividing, by the at least a processor, the portion of the plurality of fluid samples from the at least a first well into at least two wells, wherein the at least two wells comprise a time zero (T₀) well and a time one (T₁) well;
obtaining, by the at least a processor, a T₀ enumerative baseline bacterial value relating to fluid samples in the T₀ well at time T₀ using a flow cytometer;
culturing, using an incubator, the fluid samples in the T₁ well;
obtaining, by the at least a processor, a T₁ enumerative control bacterial value relating to fluid samples in the T₁ well at time T₁ using the flow cytometer; and
determining, by the at least a processor, a presence of bacteria as a function of the T₀ enumerative baseline bacterial value and the T₁ enumerative control bacterial value.

12. The method of claim 11, wherein the well plate comprises a multi-well cassette.

13. The method of claim 11, wherein distributing the portion of the plurality of fluid samples to the at least a first well comprises determining a total bacteria count of the portion of the plurality of fluid samples by enumerating the portion of the plurality of fluid samples using the flow cytometer.

14. The method of claim 13, wherein distributing the portion of the plurality of fluid samples to the at least a first well comprises adjust a dilution of the portion of the plurality of fluid samples with a growth media to a predetermined concentration as a function of the total bacteria count.

15. The method of claim 11, wherein obtaining the T₀ enumerative baseline bacterial value comprises enumerating the fluid samples in the T₀ well at time T₀.

16. The method of claim 11, wherein culturing the fluid samples in T₁ well comprises:
delivering, using a plate transport device, the fluid samples in the T₁ well to the incubator; and
returning, using the plate transport device, the fluid samples from the incubator to the fluid handling device after culturing.

17. They method of claim 11, wherein obtaining the T₁ enumerative control bacterial value comprises enumerating the fluid samples in the T₁ well at time T₁.

18. The method of claim 11, wherein determining the presence of bacteria comprises:
comparing the T₁ enumerative control bacterial value to the T₀ enumerative baseline bacterial value; and
determining a growth ratio of the portion of the plurality of fluid samples as a function of the comparison.

19. The method of claim 11, wherein the method further comprises adjusting a test enumerative bacterial value as a function of a compensator factor.

20. The method of claim 19, wherein adjusting the test enumerative bacterial value comprises:
including a known concentration of a test-enumerative compensator (TEC) particles in the fluid sample to be enumerated, wherein the TEC particles comprise known flow cytometric scatter and fluorescence characteristics;
enumerating the TEC particles with the sample enumeration by the flow cytometer; and
determining the compensator factor as a function of a comparison of a test enumerative bacterial value of the TEC particles to the know concentration of the TEC particles.
